(19) United States
(12) Patent Application Publication
(10) Pub. No.: US 2025/0249145 A1
(43) Pub. Date: Aug. 7, 2025

(54) NON-IMMUNOGENIC TISSUE AUGMENTATION FORMULATION AND METHOD FOR FABRICATION

(71) Applicant: Board of Trustees of Michigan State University, East Lansing, MI (US)

(72) Inventors: Morteza MAHMOUDI, East Lansing, MI (US); Shahriar SHARIFI, Haslet, MI (US)

(73) Assignee: Board of Trustees of Michigan State University, East Lansing, MI (US)

(21) Appl. No.: 18/855,742

(22) PCT Filed: Apr. 25, 2023
(86) PCT No.: PCT/US2023/019806

§ 371 Date: Oct. 10, 2024

Related U.S. Application Data
(60) Provisional application No. 63/334,244, filed on Apr. 25, 2022.

(51) Int. Cl. A61L 27/22; A61L 27/52; A61L 27/54; C08L 89/04

(52) U.S. Cl. CPC A61L 27/227; A61L 27/52; A61L 27/54; C08L 89/04; A61L 2300/256; A61L 2300/41; A61L 2300/416; A61L 2400/06; A61L 2430/34; C08L 2312/06

(57) ABSTRACT

Disclosed herein are tissue augmentation compositions comprising a hydrogel modified with methacrylic anhydride. The hydrogel may be composed of albumin, collagen, silk, or keratin. The composition may be used to modulate an immune response for tissue augmentation and/or as a dermal filler. The composition is non-immunogenic, non-toxic, and may be used for various biomedical applications, including passivation coatings for biomedical devices, depots, and inserts.

### CROSS-REFERENCE TO RELATED APPLICATIONS

This application claims the benefit under 35 U.S.C. § 119(e) to U.S. Provisional Application No. 63/334,244 filed on 25 Apr. 2022. The entire contents of U.S. 63/334,244 are hereby incorporated by reference in their entirety.

### GOVERNMENT RIGHTS

This invention was made with government support under DK131417 awarded by the National Institute of Diabetes and Digestive and Kidney Diseases. The government has certain rights in the invention.

### FIELD

The field of the invention relates to hydrogel compositions comprising a methacrylated protein. The hydrogel composition may be used for tissue augmentation and may modulate an immune response.

### BACKGROUND

The background description includes information that may be useful in understanding the compositions and methods described herein. It is not an admission that any of the information provided herein is prior art or relevant to the compositions and methods, or that any publication specifically or implicitly referenced is prior art.

Hydrogels have been extensively explored for various biomedical applications including tissue engineering scaffolds, drug delivery systems, and medical devices. Due to their biocompatibility, ability to hold high water content, and wide range of physicochemical properties, hydrogels can function as a biomimetic platform to reproduce in vivo microenvironment features such as extracellular matrix (ECM). Hydrogels can also support other essential biological features (e.g., facilitation of transport of nutrients and waste through their interconnected pore architecture) while facilitating design or functionalization to introduce bioactivity or smart features responding to external stimuli. Although various types of synthetic hydrogels have been developed to mimic the physicochemical and mechanical properties of natural tissues, their lack of robust cell adhesion properties has limited their safe and efficient application in tissue engineering and regenerative medicine. In contrast, natural hydrogels, which are often based on proteins, polysaccharides, or a mixture of both, or are derived from decellularized tissue, have frequently been used for various biomedical applications due to their inherent biocompatibility, bioactivity, and biodegradability.

Currently, many natural proteins (e.g., fibrin, collagen, gelatin, silk, tropoelastin, and albumin) have been used for natural hydrogel preparation. Human serum albumin (HSA) is a highly water-soluble small globular protein made of a single chain of 585 amino acids, has a molecular weight of 66,348 Da, and is the most abundant protein in blood, with an average half-life of 19 days. Produced by hepatocytes with a concentration of around 35-50 g/L in blood, this non-glycosylated endogenous protein plays a major role in the maintenance of both intravascular and extravascular colloid osmotic pressure. HSA is a well-known material for drug therapy with a long history of medical applications. As a versatile carrier protein with multiple ligand binding sites for various endogenous and exogenous molecules, it has mainly been used as i) a carrier molecule for numerous compounds such as fatty acids, metals, or drugs in drug delivery or controlled release systems; or ii) a surgical adhesive/sealant. Nonetheless, albumin hydrogels offer unique features such as biodegradability, non-immunogenicity, and biocompatibility, as well as the ability to bind to various biomolecules for their application as a biomaterial for implants or three-dimensional (3D) scaffolds for several tissue engineering applications including cardiac, orthopedic, and neural tissue regeneration.

### SUMMARY

A hydrogel composition comprising a methacrylated protein is disclosed herein. The protein may be albumin, collagen, keratin, silk, gelatin, fibrin, or tropoelastin. The amount of the methacrylated protein the hydrogel composition or the degree of methacryloylation may be adjusted or tuned as needed for a particular desired effect. The hydrogel composition may be for tissue augmentation for a therapeutic or cosmetic effect. Alternatively, the hydrogel composition may be for use in passive coatings of a biomedical device, depot, insert, or a synthetic tissue.

A hydrogel composition disclosed herein comprises a methacrylated protein prepared by dissolving an unmethacrylated protein in a buffered solution; adding methacrylic anhydride to the buffered solution to prepare a reaction mixture; incubating the reaction mixture to generate the methacrylated protein; isolating the methacrylated protein; freeze-drying the methacrylated protein; dissolving the freeze-dried methacrylated protein in deionized water to prepare a hydrogel precursor solution; adding a photoinitiator to the hydrogel precursor solution and mixing; and exposing the hydrogel precursor solution to ultraviolet light to cross-link the methacrylated protein to prepare the hydrogel composition

Use of the hydrogel composition is also described herein. The use may modulate an immune response in a subject. The use may be for tissue augmentation for a therapeutic or cosmetic effect. Alternatively, the use may be for a passive coating for a biomedical device, depot, insert, or a synthetic tissue. The modulated immune response may be demonstrated by a reduction in expression of particular cytokines.

Various objects, features, aspects, and advantages will become more apparent from the following detailed description of preferred embodiments, along with the accompanying drawings.

### BRIEF DESCRIPTION OF THE DRAWINGS

FIG. 1 is the schematic depiction of the reaction of HSA with methacrylic anhydride and the corresponding photopolymerization reaction.

FIG. 2 depicts possible reactions of tyrosine, cysteine, threonine, and serine with MAA.

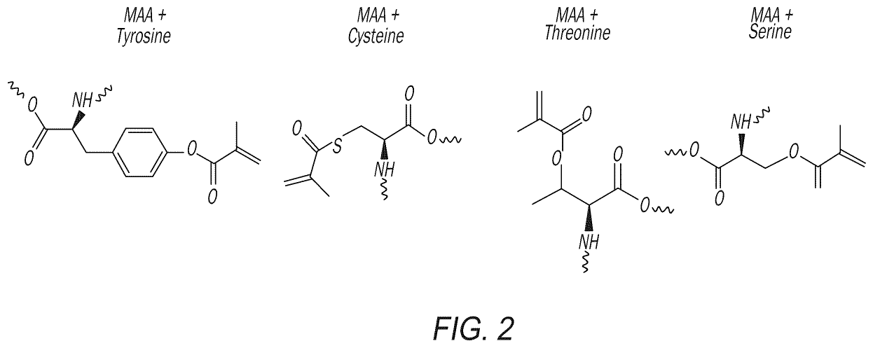

FIG. 3 depicts the size-exclusion chromatography (SEC) spectra of HSA and HSAMA (e.g., methacrylated HSA).

FIG. 4 depicts the swelling behavior of hydrogels comprising 18% (w/w) and 15% (w/w) HSAMA in water.

FIG. 5 depicts the sol fraction of hydrogels comprising 18% (w/w) and 15% (w/w) HSAMA in water.

FIG. 6 depicts an SEC spectra of pure HSA and HSAMA monomer and a chromatogram of the sol fraction after extraction of photocrosslinked samples in water.

FIG. 7 shows the weight reduction of water-swollen albumen hydrogel in enzymatic solution (n=5).

FIG. 8 is an SEC chromatogram of HSA and its degradation products (light gray), HSAMA and its degradation products (dark grey), and trypsin (hatched). The inset shows the chromatogram of pure HSA and HSAMA.

FIG. 9 is an SEC chromatogram of the supernatants from degradation medium of hydrogels (e.g., crosslinked HSAMA) in the presence or absence of trypsin. The inset shows the chromatogram of pure trypsin.

FIG. 10 depicts the cell viability of HMC3 cells treated with serial dilutions of hydrogel extract.

FIG. 11 depicts the cell viability of HMC3 cells exposed to serial dilutions of HSA or HSAMA macromers.

FIG. 12 depicts the relative spreading area of cells on the surface of a hydrogel (black bars) or glass slide (gray bars) on days 1, 3, and 5.

FIG. 13 depicts the average perimeter of HMC3 cells after a 1, 3, and 5 day culture on the surface of albumin hydrogels or glass slides.

FIG. 14 depicts the average cell Feret's diameter of HMC3 cells after a 1, 3, and 5 day culture on the surface of albumin hydrogel or glass slides.

FIG. 15 depicts the amount of 48 cytokines secreted into the media of the cells growing on the surface of the hydrogel or control group.

FIG. 16 shows the differential cytokine amount between the cells grown on TC and cells on the hydrogels.

FIG. 17 depicts the amount of 48 cytokines secreted into the media of the cells growing on the surface of the hydrogel or control group after the addition of 5 mg/mL of HSA and HSAMA.

FIG. 18 shows the differential cytokine amount between the cells grown on TC and cells on the hydrogels grown in presence of HSA and HSAMA.

FIG. 19 are microscopic images of chorioallantoic membrane (CAM) treated with PBS, HSAMA gel, or HSAMA solution.

FIG. 20 depicts the mean percentage of the vessel area, total vessel length, and mean lacunarity.

FIG. 21 depicts the percent change of vessel area, total vessel length, and mean lacunarity.

### DETAILED DESCRIPTION

### Definitions

The following definitions refer to the various terms used above and throughout the disclosure.

As used herein, all nouns in singular form are intended to convey the plural and all nouns in plural form are intended to convey the singular, except where context clearly indicates otherwise. As used herein, “and/or” includes any and all combinations of one or more of the associated listed items.

As used herein, “hydrogel” or “hydrogel composition” refers to a biphasic, porous material comprising a molecule capable of crosslinking with itself to create a three-dimensional structure. The molecule that is crosslinked may be a peptide, sugar, protein, or mixture thereof. Examples of proteins that can form hydrogels include, but are not limited to, albumin, collagen, keratin, silk, gelatin, fibrin, and tropoelastin. The crosslinking may be achieved via a chemical crosslinker, which is a compound containing amine-reactive groups (e.g., genipin and glutaraldehyde), disulfide crosslinkers, or thiol-reactive groups. The hydrogel may be crosslinked via physical approaches, such as pH-induced crosslinking or thermal crosslinking.

Alternatively, the hydrogel may be crosslinked via a photocrosslinking reaction. This approach utilizes ultraviolet light or visible light in the presence of photoinitiatiors and a molecule capable for being crosslinked, such as albumin. The photoinitiator introduces a photoreactive moiety in the albumin chain, producing three-dimensional hydrogels with controlled spatial resolution, size, and shape. The methacryloylation of albumin using methacrylic anhydride or glycidyl methacrylate in controlled pH is the most common approach for functionalization of albumin or other biomacromolecules. In the context of this invention, the hydrogel comprises a methacrylated protein.

As used herein, “methacrylated protein” refers to a protein or peptide in which one or more amino acids of the protein or peptide are functionalized and/or modified to include a methacryl moiety. The methacryloylation of the protein involves placing the methacryl moiety on an amino acid side chain capable of receiving it. Of the twenty naturally occurring amino acids, lysine, tyrosine, cysteine, threonine, and serine are capable of receiving the methacryl moiety on their side chain. The degree of methacryloylation can be determined via a chemical assay (e.g., a TNBS assay) or mass spectrometry, both of which are discussed below. Briefly, the degree of methacryloylation is calculated by determining the percentage of sites on a protein that have a methacryl moiety in relation to the number of sites on the protein that could receive the methacryl moiety. A degree of methacryloylation of 100% means that there is a methacryl moiety on all of the sites that could receive a methacryl moiety. A degree of methacryloylation of 90%, for example, means that for every 100 sites on the protein that could receive the methacryl moiety, 90 of the sites actually have a bound methacryl moiety.

As used herein, “tissue augmentation” refers to enhancing or supplementing a tissue with the hydrogel composition disclosed herein. Tissue augmentation is applicable to both therapeutic and cosmetic intentions. In therapeutic cases, tissue expansion or enlargement may be necessary to assist the proper function of tissue such as lipodystrophy in subjects suffering from HIV-associated tat loss, or repair of the vocal cord or tissue sphincters that have lost their tissue mass. Similarly, soft tissue augmentation can also be used cosmetically, such as by plastic surgeons, for people with skin aging and wrinkle formation or enlargement of body parts, including but not limited to lips or breasts, or addressing the age-related subcutaneous fat loss in areas such as the eyelid. They hydrogel composition may be used as a dermal filler. Augmentation material comprising the hydrogel composition may be administered by injection to the area for augmentation. For example, for a subject suffering lipodystrophy, the augmentation material comprising a hydrogel composition described herein may be administered to the arms, legs, face, neck, and/or chest. Alternatively, if a subject seeks a cosmetic intervention for fuller lips, the augmentation material comprising the hydrogel composition may be administered under the skin of the lip.

As used herein, “additional pharmaceutical agent” refers to a compound, molecule, peptide, or biological agent (e.g., an antibody) that may be combined with or impregnated within the hydrogel composition described herein. The additional pharmaceutical agent is not particularly limited and may be any agent that achieves a desired effect. The additional pharmaceutical agent may be one that inhibits or otherwise modulates an immune response. For example, the additional pharmaceutical agent may be one or more of an anti-inflammatory cytokine or chemokine, an antibody, an antigen-binding fragment, an anti-inflammatory agent, a non-steroidal anti-inflammatory agent (NSAID), and an anti-angiogenic agent.

As used herein, “immune response” represents the action of one or more components of an immune system in reaction to one or more stimuli. The immune response may occur within a body of an animal (e.g., a human), outside the body of an animal (e.g., an ex vivo tissue), or in an in vitro environment that mimics the immune response. The immune response includes both the innate and the adaptive immune systems. Modulating an immune response includes both enhancing an immune response or inhibiting an immune response. Enhancing an immune response may include increasing expression and/or release of pro-inflammatory cytokines (e.g., IL-1, TNF-α, IL-6, and IFN-γ), increasing the inflammatory activity of immune cells, decreasing expression and/or release of anti-inflammatory cytokines (e.g., IL-4, IL-13, IL-10, and TGF-3), and/or decreasing the inflammatory activity of regulatory cells. Inhibiting an immune response may include decreasing expression and/or release of pro-inflammatory cytokines, decreasing the inflammatory activity of immune cells, increasing expression and/or release of anti-inflammatory cytokines, and/or increasing the activity of regulatory cells.

As used herein, “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In certain embodiments, the subject can be human (e.g., adult male, adult female, adolescent male, adolescent female, male child, female child) under the care of a physician or other health worker. In certain embodiments the subject may not be under the care of a physician or other health worker. The subject may have undergone surgery, received orthopedic treatment, received ophthalmic treatment, or suffering from injury or chronic disease. Alternatively, where the subject is a laboratory mammal, the hydrogel composition may be provided to the laboratory mammal to achieve a scientific understanding rather than a clinical benefit.

As used herein, “passivation” refers to coating a material with a hydrogel composition described herein such that the material does not induce an adverse response from a subject, such as an adverse immune response. The coated material may be a biomedical device, depot, insert, or synthetic (e.g., artificial) tissue that mimics the structure, shape, and/or function of an endogenous organ, tissue, cell, body part, or portion thereof. Passivation may reduce or inhibit an immune response to the device, depot, insert, or synthetic tissue by preventing the subject's immune system from recognizing the device, depot, insert, or synthetic tissue. Alternatively, the coated material may provide anti-inflammatory agents into the local environment, such as by release of pharmaceutical agents located within the hydrogel composition that coats the device, depot, insert, or synthetic tissue, or by degradation of the hydrogel composition itself to downregulate the immune response.

Albumin, a non-adhesion protein, is widely involved in surface passivation applications; albumin gels, however, are generally not known to support cell adhesion. For example, cell attachment to surfaces with adhesive molecules is an indirect process that progresses through rapid adsorption of serum proteins on the substrate acting as a spacer between the cells and substrate. Bovine serum albumin (BSA) coating has been shown to reduce the attachment of the microglial cells, possibly through establishing a low protein binding surface. Albumin may have different conformations or even some degree of denaturation depending on its production method and purity level, both of which affect its passivation efficiency. For example, a fat-free BSA coating created by heat shock fractionation on the surface of silica nanoparticles prevented absorption of proteins from FBS up to 80%. The passivation efficacy of full-fat BSA produced through cold ethanol fractioning followed by heat shock fractionation was about 40%.

Adhesion of the cells to albumin may imply some degree of denaturation, which would promote absorption of adhesive protein from the serum onto the hydrogel surface, mediating cellular attachment. The degree of alteration in albumin conformity and especially the loss of α-helix is directly proportional to platelet adhesion on the surface of biomaterials. This could be the prevalent mechanism of action responsible for the attachment of the cells to albumin hydrogels.

### Hydrogel Compositions

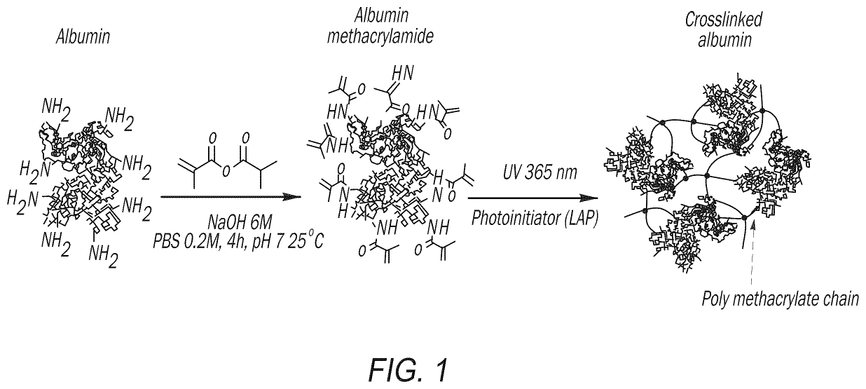

The hydrogel compositions described herein may comprise a modified protein or peptide, such as a methacrylated protein, wherein a methacryl moiety is bound to a plurality of sites within the protein that are capable of receiving a methacryl moiety. An example of the methacrylated protein within the hydrogel composition can be seen in FIG. 1. The protein is not particularly limited and may be any protein or peptide capable of crosslinking to such a degree as to form a hydrogel. Examples of proteins suitable for forming a hydrogel include, but are not limited to, albumin, collagen, keratin, silk, gelatin, fibrin, and tropoelastin. In particular, the protein may be albumin. The albumin may be naturally occurring (e.g., human albumin, bovine albumin) or synthetic or recombinant albumin (e.g., albumin prepared from bacteria). In a preferred embodiment, the albumin is a human albumin, such as human serum albumin.

The hydrogel comprises a sufficient amount of protein, as measured by weight percentage (% (w/w)), to hold a high water content, retain a shape for a sufficient period of time, and maintain a sufficiently porous architecture. For example, the hydrogel composition may comprise between about 5% (w/w) and about 25% (w/w), between about 10% (w/w) and about 20% (w/w) or between about 15% (w/w) and about 18% (w/w). Alternatively, the hydrogel composition may comprise about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), or about 25% (w/w) of the methacrylated protein (e.g., methacrylated albumin). In particular, the hydrogel composition may comprise about 11% (w/w), about 15% (w/w), or about 18% (w/w) methacrylated protein.

The degree of methacryloylation of the methacrylated protein may be adjusted (e.g., tuned) to induce a desired degree of immunogenicity or immunosuppression. For example the degree of methacryloylation of the methacrylated protein may be greater than about 70%, greater than about 75%, greater than about 80%, greater than about 85%, greater than about 90, greater than about 95%, or greater than about 99%. In some embodiments, the degree of methacryloylation may be 100%.

The hydrogel composition may be used for tissue augmentation. For example, the hydrogel composition may be used for tissue augmentation for a therapeutic effect. The therapeutic effect is not particularly limited and refers to any effect in which a subject has a disease, disorder, or condition, or is at risk for a disease, disorder, or condition, and the hydrogel composition is administered to treat, alleviate, cure, prevent, and/or reduce the risk of the disease, disorder, or condition. For example, the subject may suffer from lipodystrophy, damaged vocal cords, or damaged tissue sphincters and the hydrogel composition is administered to treat, alleviate, or cure the lipodystrophy, damaged vocal cords, or damaged tissue sphincters. In another embodiment, the hydrogel composition may be used in a method for treating a disease, disorder, or condition in a subject in need thereof. The method includes administering to the subject a therapeutically effective amount of the hydrogel composition. The hydrogel composition may be formulated as above, wherein the amount of methacrylated protein in the hydrogel and/or degree of methacryloylation is adjusted to a desired degree.

Alternatively or additionally, the hydrogel composition may be used for a cosmetic effect. The cosmetic effect is not particularly limited and refers to any effect in which the subject seeks to change an aspect of their body for non-therapeutic reasons. For example, the subject may seek to enlarge a body part (e.g., lips or breasts), replace age-related subcutaneous fat loss, remove wrinkles, and/or as a dermal filler.

Alternatively, the hydrogel composition may be used as a passivation coating for a biomedical device, depot, insert, or synthetic tissue.

Where the hydrogel composition is used for tissue augmentation (either for therapeutic use or for cosmetic use) or as a passivation coating, for a biomedical device, depot, insert, or synthetic tissue, the hydrogel composition may be adjusted as appropriate to accommodate the purpose for which the hydrogel composition is to be used. For example, the amount of the methacrylated protein (e.g., the weight/weight percentage of the methacrylated protein in the hydrogel) may be adjusted to include a particular concentration to induce a desired effect, such as to 15% (w/w) or 18% (w/w). Alternatively, the degree of methacryloylation of the methacrylated protein may be adjusted as desired. For example the degree of methacryloylation may be at about 95% or about 100%.

Additionally or alternatively, the hydrogel may be in the form of microparticles. The microparticles may be regularly or irregularly shaped. In a specific embodiment, the microparticles are irregularly shaped. The size of the microparticles is not particularly limited and may be between about 0.01 to about 100 μm in diameter. This size may be an average size. In particular, the microparticles may be between about 10 and 40 μm or between about 1 and about 100 μm in diameter. Alternatively, the microparticles may have a diameter of about 0.01 μm, about 0.1 μm, about 1 μm, about 10 μm, or about 100 μm. Furthermore, the hydrogel formulations may comprise microparticles that are in a multimodal distribution, wherein the hydrogel comprises a mixture of size of microparticles. For example, the hydrogel may comprise a population of microparticles having an average size of about 30-40 μm and microparticles having an average size of about 10-15 μm.

The hydrogel composition may be a bulk hydrogel in a non-particulate form. Such compositions are suitable for injection. Alternatively, the bulk hydrogel may be mixed or combined with microparticles. The microparticles may have an average diameter as described above. Further, the hydrogel composition comprises a combination of a hydrogel comprising the methacrylated protein described above, a bulk hydrogel from another carrier gel, such as carboxymethyl cellulose, and microparticles, such as microparticles form albumin hydrogels. The particle size of the microparticles is described above. The ratio of the particulate phase to the non-particulate phase is not particularly limited. For example, the ratio may be in a range of about 1:99 to about 30:70. The ratio may be a particular ratio, such as about 1:99, about 5:95, about 10:90, about 15:85, about 20:80, about 25:75, and about 30:70.

Additionally or alternatively, the hydrogel composition may further comprise one or more additional pharmaceutical agents. The one or more additional pharmaceutical agents are not particularly limited. For example, the one or more additional pharmaceutical agent may be an anti-inflammatory cytokine, an antibody, an antigen-binding fragment, an anti-inflammatory agent, an NSAID, an anti-angiogenic agent, or combinations thereof. The additional pharmaceutical agents may be present in a therapeutically effective amount, and may be adjusted as necessary.

The hydrogel composition described herein may be prepared by: dissolving an unmethacrylated protein in a buffered solution; adding methacrylic anhydride to the buffered solution to prepare a reaction mixture; incubating the reaction mixture to generate the methacrylated protein; isolating the methacrylated protein; freeze-drying the methacrylated protein; dissolving the freeze-dried methacrylated protein in deionized water to prepare a hydrogel precursor solution; adding a photoinitiator to the hydrogel precursor solution and mixing; and exposing the hydrogel precursor solution to ultraviolet light to cross-link the methacrylated protein to prepare the hydrogel composition. The protein in the hydrogel composition prepared as above may be albumin, collagen, keratin, silk, gelatin, fibrin, or tropoelastin. Preferably, the protein in the hydrogel composition is albumin. The hydrogel composition may be prepared though an incubation at room temperature at a neutral or slightly basic pH. A neutral pH is a pH of about 7. The solution is slightly basic if it has a pH above 7 but less than 10, for example a pH of 7.5, 8, 8.5, 9, or 9.5. The hydrogel composition may be prepared including a step for isolating the methacrylated protein in isolation by dialysis in deionized water. The amount of water provided may be in an amount sufficient to prepare a hydrogel composition comprising between about 5% and about 25% (w/w) of the methacrylated protein.

Alternatively, the amount of water may be sufficient to prepare a hydrogel composition comprising between about 10% and about 20% (w/w), between about 15% and about 18% (w/w) or any specific amount of methacrylated protein within these ranges. In particular, the hydrogel composition may be prepared to comprise about 11% (w/w), about 15% (w/w), or about 18% (w/w) of the methacrylated protein. The hydrogel composition may be prepared such that at least 20%, at least 30%, at least 40%, at least 50%, at least 60%, at least 70%, at least 80%, at least 90%, at least 95%, at least 99%, or all (e.g., 100%) of the functional groups on the unmethacrylated protein that are reactive to methacrylic anhydride are reacted.

The photoinitiator used in the preparation of the hydrogel composition is not particularly limited, but may be lithium phenyl-2,4,6-trimethylbenzoylphosphinate (LAP), 2-hydroxy-4′-(2-hydroxyethoxy)-2-methylpropiophenone (Irgacure 2959), or ascorbic acid. When ultraviolet light is used to activate the photoinitiator, it may be provided in an intensity of between about 1 to about 20 mW/cm², such as between about 6 and about 10 mW/cm². In a particular embodiment, the ultraviolet light intensity is about 6 mW/cm².

### Methods and Uses

The hydrogel compositions described herein may be for use for tissue augmentation. In one embodiment is a method for modulating an immune response in a subject in need thereof. The method may comprise administering to the subject a therapeutic amount of the hydrogel composition comprising a methacrylated protein. The relative amount of the methacrylated protein within the hydrogel composition may be adjusted in light of the therapeutic or cosmetic effect to be achieved. The method may comprise injecting the hydrogel composition directly to the desired site. The method may reduce expression of one or more cytokines and/or growth factors. In particular, the method may reduce expression of IL-6, IL8, and/or VEGF.

An alternative embodiment is a use of the hydrogel composition described herein for modulating an immune response. The relative amount of the methacrylated protein within the hydrogel composition may be adjusted in light of the therapeutic or cosmetic effect to be achieved. The use may comprise injecting the hydrogel composition directly to the desired site. The use may reduce expression of one or more cytokines and/or growth factors. In particular, the use may reduce expression of IL-6, IL8, and/or VEGF.

The methods and uses described herein may further comprise including one or more additional pharmaceutical agents in the hydrogel composition. The one or more additional pharmaceutical agents can be selected based on the specific therapeutic or cosmetic effect to be achieved. For example, the hydrogel may comprise albumin microparticles which may be used for subcutaneous injection to induce collagen formation for wrinkle removal.

### Examples

The following example are provided to further illustrate the hydrogel composition disclosed herein but should not be construed as in any way limiting its scope.

### Example 1: Preparation of Methacrylated Albumin

Human serum albumin (HSA), fatty acid-free, was obtained from Cone Bioproducts (Seguin, TX, USA). Dialysis tubes (MWCO=6-8 kDa) were from Spectrum Laboratories Inc (Billerica, MA, USA). All other materials were purchased from Sigma-Aldrich unless otherwise stated. 2,4,6-trinitrobenzene sulfonic acid (TNBS), methacrylic anhydride (MAA), and lithium phenyl-2,4,6-trimethylbenzoylphosphinate (LAP) were used as received.

Synthesis of methacrylated albumin (HSAMA): Methacrylated albumin was synthesized by dissolving 10 g of HSA in 150 mL of 0.2 M phosphate buffer at pH 7. Methacrylic anhydride, in 5 molar times excess compared to theoretical free lysine residues of HSA, was added under magnetic stirring with 300 RPM. The reaction was continued for 4 h at room temperature at neutral or slightly basic pH by adding NaOH during the reaction course to prevent denaturation and agglomeration due to acidification. The reaction mixture was then kept at 4° C. overnight to crystallize methacrylate sodium salt, a byproduct of the reaction. The salt was then removed by vacuum filtration. The reaction solution was introduced into dialysis tubes and dialyzed in the dark in 10 L of deionized sterile water over 3 days at room temperature. The water was refreshed every 12 h. The pH of the final solution was then adjusted by the addition of sodium bicarbonate (1 M) to obtain methacrylated albumin solution with a pH range of 7.2-7.4. The final purified product was then snap-frozen with liquid nitrogen and freeze-dried for 5 days to obtain fluffy methacrylated albumin cakes with a yield of 84%. The product was then stored at −80° C. until use. To evaluate the repeatability of the synthesis, two independent batches were synthesized and named M9 and M10. After methacryloylation of HSA, the degree of methacryloylation was determined by quantifying the remaining free amino groups in HSA utilizing a TNBS assay or by matrix-assisted laser desorption/ionization time-of-flight (MALDI-TOF) mass spectrometry.

Determination of degree of methacryloylation by TNBS assay: The TNBS assay was performed by dissolving 2.0 mg of methacrylated albumin in 0.1 M sodium bicarbonate (pH 8.5). Then 250 μL of 0.01% (w/v) TNBS was added to 500 μL of the sample solution in 48 well-plates and incubated at 37° C. for 2 h. The reaction was stopped by the addition of 250 μL of 10% SDS and 125 μL of 1 M HCl followed by mixing. The absorbance of the samples was measured in a microplate reader (SpectraMax® ID5, Molecular Devices) at 335 nm. A serial dilution of glycine in 0.1 M sodium bicarbonate at a concentration of 2.5, 5, 7.5, 10, 15, 17, and 20 μg/mL was prepared, and the standard curve was obtained to determine the NH₂ group concentration. The degree of methacryloylation was then calculated according to the following equation, where C_{NH}_{₂}HSA is the concentration of NH₂ groups in HSA and C_{NH}_{₂}HSAMA is the concentration of the NH₂ groups in methacrylated albumin.

$$Degree of methacryloylation = ( \frac{{C}_{{NH}_{2}} HSA - {c}_{{NH}_{2}} HSAMA}{{c}_{{N}_{{H}_{2}}} HSA} ) \times 100 \qquad ( 1 )$$

Determination of degree of methacryloylation by MALDI-TOF-MS: MALDI-TOF analysis was performed using Shimadzu Axima cfr-plus. Prior to analysis, both HSA and HSAMA were first desalted against ultrapure water using H₂O+0.1% trifluoroacetic acid (TFA) as solvent and Bond Elut OMIX C4 (Agilent Technologies) pipette tips. They then were eluted from the tip using acetonitrile (ACN)/H₂O (50:50 (v/v) with 0.1% TFA). The samples for MALDI analysis were prepared by the dried-droplet method: 0.6 μL of 10 mg/mL sinapic acid (in ACN/H₂O (50:50 (v/v) with 0.1% TFA) was loaded on the MALDI slide, and after drying, 0.6 μL of protein elute from Bond Elut OMIX C4 was loaded on top of the dried matrix spot. Analysis of HSA and HSAMA were conducted with positive ionization at an acceleration voltage of 20 kV. The spectra were obtained over the m/z range from 0 to 500 kDa. The number of functional methacrylated groups was then calculated using following equation, where the M HSMA is the molecular weight of methacrylated albumin and M HSA is the molecular weight of albumin. Since lysine is the major reactive amino acid, M Methacrylamide was considered the molecular weight of methacrylamide monomer (84.12 Da).

$$Number of modified amino acid groups in HSAMA = ( \frac{M HSAMA - M HSA}{M Methacrylamide} ) \times 100 \qquad ( 2 )$$

Determination of modified amino acid residues in HSAMA by LC-MS/MS: Since MAA could potentially react with both amino (NH₂) and hydroxyl (OH) groups, the amino acid sequences in HSAMA were analyzed utilizing LC-MS/MS and evaluated whether e-ammonium group (NH₃⁺) containing amino acids such as Lys and amino acids containing nucleophilic chemical groups, such as Cys, Thr, Ser, and Tyr react with MAA. Therefore, HSA and HSAMA samples were digested according to our previous protocol with slight modifications, and each sequence was identified using LC-MS/M. To further assess the purity of HSA and HSAMA, structure and aggregation state in buffer, we used Fast protein liquid chromatography (FPLC), a form of size exclusion chromatography often used to analyze or purify mixtures of proteins was used to assess the purity of HSA and HSAMA.

Sample preparation for LC-MS/MS: After in silico simulation of digestion (web.expasy.org/peptide_mass), we chose two proteases for HSA digestion, i.e., trypsin and endoproteinase GluC (S. aureus Protease V8), to improve sequence coverage in protein characterization. Briefly, 50 μg of control HSA and HSAMA from two consecutively synthesized batches (M9 and M10) were dissolved in 50 mM ammonium bicarbonate buffer (pH 8.5) with 1 M urea. Samples were reduced with dithiothreitol (DTT) to a final concentration of 10 mM for 1 h at room temperature. Afterwards, iodoacetamide (IAA) was added to a final concentration of 50 mM. The samples were incubated at room temperature for 1 h in the dark, and the reaction was stopped by the addition of 10 mM DTT. Samples were enzymatically digested either with trypsin or GluC (both 1:75 w/w protease:protein) separately, then acidified with TFA, cleaned using Sep-Pak (Waters; Cat #WAT054960), and dried using a DNA 120 SpeedVac™ concentrator (Thermo Fisher Scientific).

LC-MS/MS data acquisition and analysis: Samples digested with trypsin or GluC were separately loaded with buffer A (0.1% formic acid (FA) in water) onto a 50 cm EASY-Spray column (75 μm internal diameter, packed with PepMap C18, 2 μm beads, 100 Å pore size) connected to a nanoflow UltiMate 3000 UPLC system (Thermo Fisher Scientific) and eluted in 60 min long linear organic solvent gradient from 4 to 26% B (B: 98% ACN, 0.1% FA, 2% H₂O) at a flow rate of 300 nL/min. Mass spectra were acquired with a Q Exactive HF hybrid quadrupole orbitrap mass spectrometer (Thermo Fisher Scientific, Bremen, Germany) in the data-dependent mode acquiring full mass spectra at 120,000 resolution and fragment ion spectra at 30,000 (@200 m/z), in the mass range from m/z 375 to 1500. Peptide fragmentation was performed via higher-energy collision dissociation (HCD) with normalized collision energy set at 28% following isolation of precursor ions with 1.4 Th.

Acquired raw data files were analyzed using Proteome Discoverer v2.5 (Thermo Fisher Scientific) with Mascot Server v2.5.1 search engine (Matrix Science Ltd., UK) against human serum albumin (P02768). Proteases were selected as trypsin or GluC (with selective cleavage after Glu only), allowing three missed cleavages. Precursor and fragment ion mass tolerances were set to 10 ppm and 0.02 Da, respectively. Variable modifications were defined as acetylation on N-termini, carbamidomethylation on Cys, deamidation on Asn and Gln, as well as methacryloylation on Lys or Lys, Cys, Ser, Thr, Tyr. Initial search results were filtered with 5% FDR using Fixed Value PSM Validator node and modification probability was calculated with the IMP-ptmRS node in Proteome Discoverer.

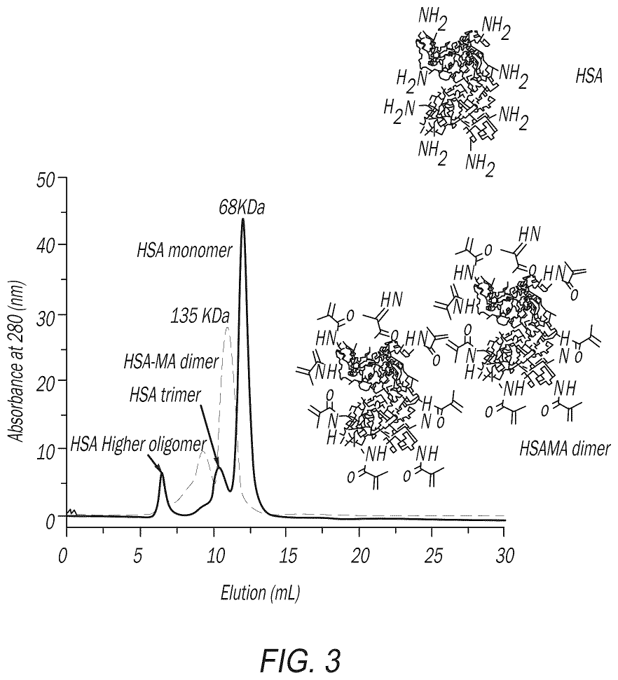

The chromatogram of HSA and HSAMA is shown in FIG. 3 and was in agreement with the MALDI-TOF-MS results (not shown). In the solution state, HSA was mainly in monomeric form; however, it also contained a small number of higher oligomers.

Fabrication of HSAMA hydrogels: To obtain albumin hydrogels, the first freeze-dried HSAMA macromer was dissolved in deionized water at 12, 15, and 18% (w/w). Following complete dissolution, the LAP photoinitiator at 1% (w/w) of macromer was added to each solution. The reaction was gently mixed, and the solution was then poured into a circular silicon mold with a diameter of 18 mm and a height of 4 mm. The hydrogel precursor was then crosslinked in the UV crosslinker oven under argon blanket at 365 nm (Boekel Scientific 234100-2-USA) at intensity of circa 6 mW/cm² for 10 min. The hydrogel specimens with a diameter of 5 mm and height of 4 mm were punched from crosslinked albumin disc and were used for swelling, biodegradation, and biocompatibility study.

Swellinq study of HSAMA hydrogels: Following crosslinking, the HSAMA hydrogels were weighed (Wi) and then transferred to a glass vial containing 20 mL of deionized water and allowed to swell at room temperature for 3 days till constant weight (Ws) was achieved. The prepared hydrogels (n=5) were dried in an oven at 37° C. until samples were completely dry and reached contact weight (Wd). Following complete hydration, hydrogels were dried at 37° C. and weighed again to obtain sol-free weight (Wf). The swelling degree and sol fraction were calculated from the following equations.

$$Swelling degree ( \% ) = ( \frac{Ws - Wd}{Wd} ) \times 100 \qquad ( 3 )$$

$$Sol fraction ( \% ) = ( \frac{Wd - Wf}{Wd} ) \times 100 \qquad ( 4 )$$

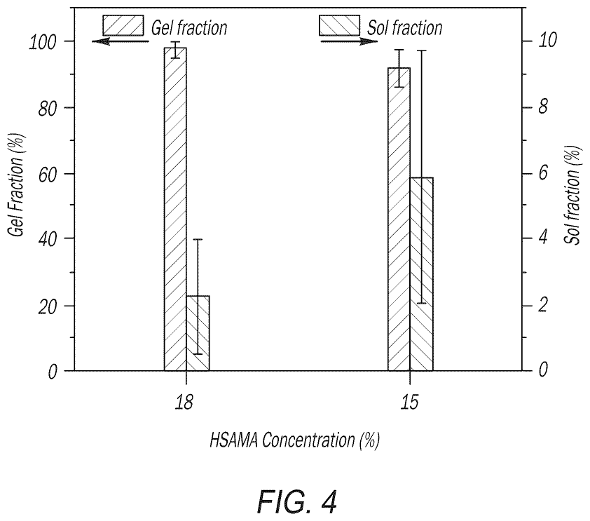

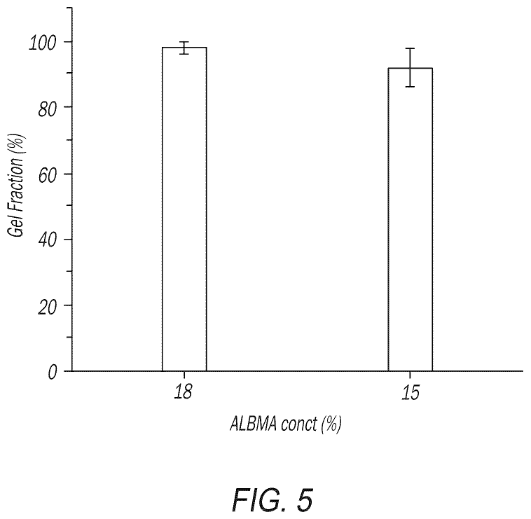

The degrees of swelling of the 15% and 18% (w/w) HSAMA hydrogels in water are shown in FIG. 4. The sol factions of the 15% and 18% (w/w) hydrogels in water are shown in FIG. 5. A low concentration of HSAMA (11% w/w) yielded a soft, putty-like hydrogel, and since handling was not possible, the swelling ratio and gel fraction were not measured. Albumin hydrogels reached their equilibrium swelling ratio, which was dependent on the initial concentration of the macromer. The degree of swelling of the hydrogels is regulated by their crosslinked density, which depends on the concentration of cross-linkable groups 62. Hence the crosslinked density can be tuned by either varying the degree of methacrylate modification in the protein or altering the concentration of polymerizable groups.

Compared to recent reports on photocrosslinked BSA hydrogels (e.g., Lantigua, Soft Matter, 2020 16(40), 9242-9252; Feracci, et al. ACS Appl Bio Mat, 2020, 3(2), 920-934), the water uptake of the disclosed hydrogel at low concentration (15% w/w) was about two times higher. However, increasing the HSAMA concentration to 18% w/w yielded a much denser network with a 2.4-fold reduced swelling ratio. The physicochemical properties of hydrogels may dependent on polymerization conditions such as the photoinitiator system, light intensity, irradiation time, amongst other variables. Here, LAP was used as the photoinitiator, whereas Irgacure 2959 is also used in the art. However, studies with LAP and Irgacure 2959 show that the swelling ratio of hydrogels is not affected by the type of the initiator. The hydrogel in this example was prepared using much lower UV light intensity of about 6 mW/cm² compared to 150 and 700 mW/cm².

Examination of composition of sol fraction and degradation products using size exclusion chromatography (SEC): The sol fraction composition from the swelling study as well as the degradation study was analyzed by size exclusion chromatography. Briefly, 200 μL of the sample was injected onto a Superdex 200 analytical column (GE, cat #17-108801) pre-equilibrated with 1×PBS (Gibco, cat #10010-023) using an Akta Pure FPLC system (Cytiva) with a flow rate of 0.4 mL/min. The calibration curve was determined by injection of 200 μL of gel filtration standards (BioRad, cat #151-1901), which includes lyophilized mixture of thyroglobulin, bovine γ-globulin, chicken ovalbumin, equine myoglobin, and vitamin B12, covering molecular weight range of 1,350 Da to 670 kDa.

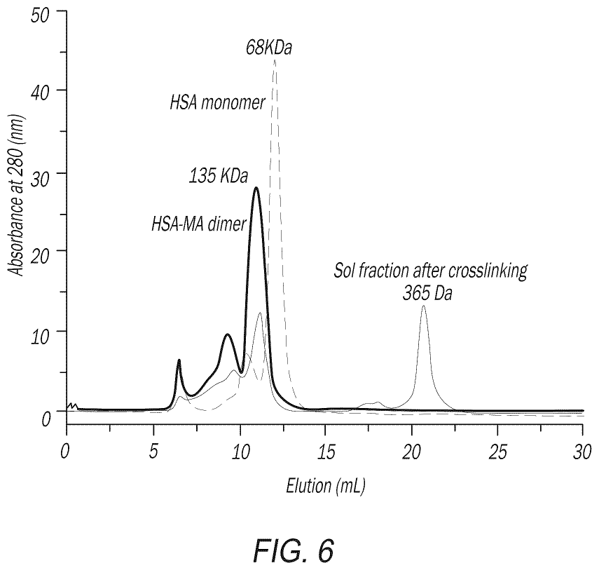

Due to the importance of the sol fraction, especially to the biological and biocompatibility response, the hydrogels were analyzed by SEC. The sol fraction (from 15% w/w sample) consisted of non-reacted HSAMA macromer and low-molecular-weight fragments with a peak molecular weight of 356 Da. See FIG. 6. Long-wave UV (UVA) at low intensities and exposure time may not have sufficient energy to induce chain secession in the protein. In the presence of a photoinitiator, the generated radicals can induce photodegradation or change the physicochemical properties of proteins. For instance, BSA has been known to release free amino acids when exposed to oxidizing agents such as hydroxyl radicals, which can be formed when water is present in the photopolymerization medium. Although not analyzed, the composition of this sol fraction likely comprises albumin oxidation products and amino acids, as suggested by the molecular weights of residues determined by SEC shown in FIG. 6.

The samples turned to pale yellow after UV irradiation (not shown). Though LAP is known to exhibit low yellowing, the yellow discoloration is probably due to the formation of chromophores such as hydroperoxide. The photo yellowing was reversible, and the samples were colorless after a few hours of UV exposure or soaking in water.

Hydrogel porosity: The porosity of a hydrogel is a crucial factor that not only influences its mechanical properties but also regulates the diffusion of nutrients, waste removal from encapsulated cells, and its biodegradation rate as well as tissue ingrowth and vascularization. Depending on the type and architecture of monomer or macromer, hydrogel porosity can be tuned by changing the molecular weight of the macromer or degree of side chain functionalization. For example, reducing molar mass in the case of telechelic oligomer end-functionalized with acrylate or methacrylate groups results in lower hydrogel porosity. Alternatively, in the case of side-chain-functionalized precursors, hydrogel porosity may be controlled by the degree of functionalization as shown for BSA or other hydrogels. See Lantigua, Soft Matter, 2020 16(40), 9242-9252. Hydrogel porosity can also be controlled by macromer concentration. Three different concentrations of hydrogels were analyzed to explore the effect of HSAMA concentration on hydrogel porosity. The hydrogels were dried with a critical point dryer to maintain the pore structure intact, and the pore structures as observed by SEM were uniform and fairly distributed along with the matrix. Hydrogels with low concentrations of HSAMA (e.g., 11% (w/w)) had a thin wall and showed numerous small pores with an average size of 10.13±3.79 μm. However, increasing the concentration of HSAMA to 15 and 18% (w/w) thickened the cell walls, and the hydrogel average pore size was also increased to 20.04±5.45 and 44.9±21.6 μm, respectively. SEM images confirmed the resulting hydrogel was less porous compared to hydrogels with lower concentrations of HSAMA. Data not shown.

Scaffold architecture and pore size can directly affect cell behavior and especially cellular proliferation and differentiation, as they provide mechanical and biological cues that regulate cell-matrix interactions. The optimum value for scaffold pore size is cell-specific and also depends on the physicochemical priorities of the material. For example, crosslinked collagen-glycosaminoglycan scaffold with pore size of 20-125 μm has been reported to support partial morphogenesis of skin in a Guinea pig model. Therefore, HSAMA concentration can be an effective tool to adjust the physicochemical properties of hydrogel, especially pore size and distribution, to meet the requirements of cell proliferation and infiltration.

Degradation study: Trypsin was used for enzymatic degradation of HSA and HSAMA. Briefly, 5 mg of each protein was dissolved in 1 mL of 0.25 mg/mL of a solution of bovine pancreas trypsin (>10,000 BAEE units/mg protein) treated with L-1-tosylamide-2-phenylethyl chloromethyl ketone (TPCK) for 24-48 h at 37° C. The degradation and fragmentation patterns of both HSA and HSAMA were then analyzed by size exclusion chromatography. Similarly, hydrolytic and enzymatic degradation of albumin hydrogels were also evaluated in water and trypsin solution. Hydrogels for degradation testing were fabricated using the same protocol as described above; however, only one concentration was prepared (15%, w/w). Before the degradation test, hydrogels (n=5) were put in excess deionized water for 48 h to extract the sol fraction and leachables. Hydrogels were then blotted with tissue paper and weighed. Cylindrical hydrogels (D, H 5 mm) were then degraded enzymatically using 1 mL trypsin solution. At predetermined time intervals, the hydrogels were extracted from the solution and weighed after blotting. Similarly, the degradation of the hydrogel in pure water at 37° C. was monitored. At a predetermined time interval, the protein concentration in the degradation solution was measured by a nanodrop spectrophotometer (NanoDrop 8000, Thermo Scientific, USA). The composition of degradation superannuate was also analyzed by SEC study. The degradation study was replicated for three samples in the course of the degradation study. Trypsin EDTA for cell culture (0.25%, porcine trypsin, Sigma) in Hanks' Balanced Salt Solution at 37° C. was used for preliminary degradation study.

When designing hydrogel scaffolds for tissue engineering, hydrogel degradation rate plays a crucial role, since there should be a match between scaffold replacement and tissue regeneration. In addition, hydrogels and their degradation products should elicit minimal foreign body response. Degradation of albumin hydrogels is dependent on the preparation method, and the degree of protein modification can determine its degradation rate and biocompatibility. Natural and endogenous HSA has a half-life of 19 days and can degrade in any tissue through proteolytic enzymes. However, its degradation mainly takes place in the liver and kidney. Synthetic albumin hydrogels, on the other hand, can display a wide variety of physicochemical characteristics, as well as a wide range of degradation profiles ranging from fast-dissolving gels to long-lasting hydrogels. In fact, the physical parameters of gels such as microstructure, porosity, and elastic modulus can influence the behavior of macrophages and other phagocytic cells and hence alter the degradation rate of synthetic albumin-based gels. For example, while BSA gels prepared by pH-induced BSA significantly degraded after 4 weeks of subcutaneous implantation in Sprague-Dawley rats, their thermally denatured BSA counterparts showed no signs of degradation. Alterations in albumin structure or its modifications may also trigger its biodegradation or recycling via receptor-mediated endocytosis followed by lysosomal degradation through Gp18 and Gp30 receptor expressed on endothelial cell membranes of liver cells or peritoneal macrophages. In fact, it has been shown that albumin modified by common crosslinker molecules such as formaldehyde or maleic anhydride have much higher affinity for Gp18 and Gp30 compared to native albumin.

Hydrogels were evaluated by SEC, spectroscopy, and gravimetry for the presence of degradation products from bovine trypsin and in vitro enzymatic degradation of 100% methacrylated HSA macromer. Porcine-derived trypsin-EDTA was initially used in cell culture (Gibco). However, preliminary SEC studies showed that this trypsin was a mixture of components, probably a cocktail of proleptic enzymes. Hence, to facilitate the identification of the degradation products, pure bovine trypsin, which was TPCK-treated to inhibit the chymotrypsin, was used.

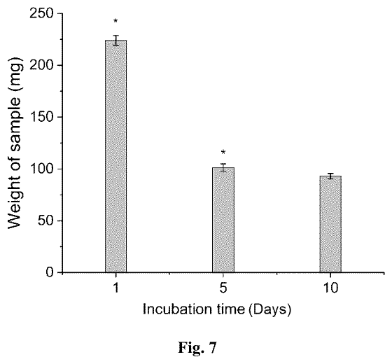

To eliminate the interference of the sol fraction in the degradation study, the gels were first extracted in water to ensure that any weight reduction was the result of degradation (FIG. 7). As expected, hydrogel mass was reduced 55% after 5 days of incubation in trypsin solution. The protein concentration of the supernatant was also measured by UV spectroscopy (Nanodrop). A 1.9-fold increase in protein concentration was observed, which provided further evidence for ongoing proteolytic and degradation activity. However, the degradation rate decreased over the study course, and after five days' incubation with the hydrogel, mass was reduced by only 3%, culminating in a total weight reduction of 58% after 10-day incubation in tryptic solution. Similarly, the protein concentration of the solution was only slightly increased (17%) after five additional days of incubation. These results show that hydrogels synthesized with 100% methacrylated HSA may have increased resistance to enzymatic degradation, probably due to higher crosslink density or reduced reactivity toward proteolytic enzyme.

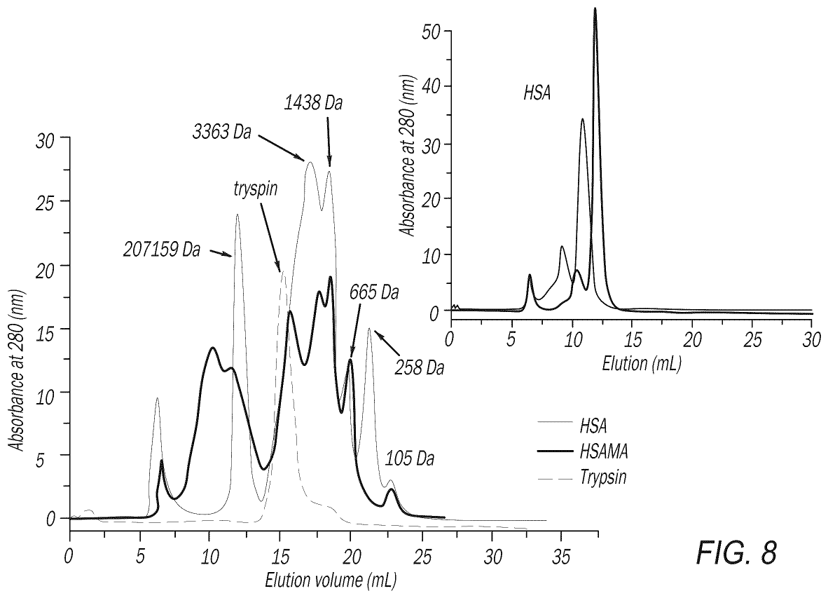

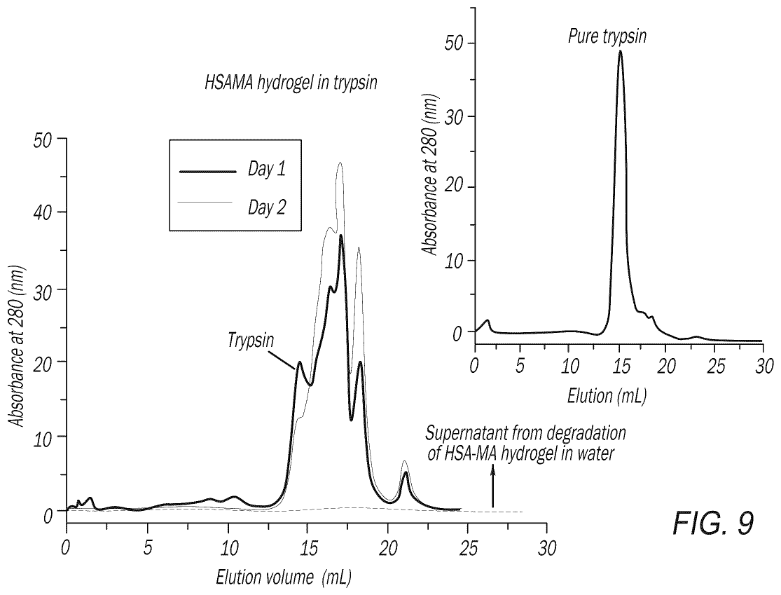

To further explore the effect of methacryloylation on enzymatic degradation of the HSA, the degradation supernatants of HSAMA and the hydrogel were analyzed by SEC (FIGS. 8 and 9, respectively). The chromatogram of trypsin-treated HSA showed five short fragments with a molar mass of 3363, 1438, and 665 Da and two monomeric components with molar mass of 258 and 105 Da. Some intact HSA was also detected in the chromatogram. Trypsin-treated HSAMA showed a degradation pattern relatively similar to that of HSA, and fragments with a molar mass of 3363, 1438, and 665 Da were common to both HSA and methacrylated HSA. However, the degradation fragments were also composed of very large aggregates, and monomeric fragments with a molar mass of 258 Da were absent from the spectra of HSAMA. These results support the notion that methacryloylation of albumin affects its susceptibility to tryptic digestion. The trypsin binding pocket is suitable for the ionic bonding of long side chain and positively charged residues such as lysine and arginine. Hence, trypsin exclusively cleaves the C-terminal of these residues. Methacryloylation mainly targets lysine residues and removes the positive charge from the N-terminal amino group. Moreover, methacryloylation also alters the hydrophobic characteristic of HSA. Therefore, it is possible that trypsin cleavage is reduced or even blocked. This finding is corroborated by the results of other studies on BSA, indicating that reduced degradation of fully methacrylated albumin gel may be partially related to altered enzyme activity on the hydrogel.

SEC analysis of degradation products from hydrogels (FIG. 9) demonstrated that the fragmentation pattern was similar to that of HSA, and the chromatogram showed four oligomeric and monomeric peptide fragments with a molar mass of 2154, 1400, 657, and 100 Da. The degradation product from day 1 was also similar to day 2, indicating continuous proteolytic action of trypsin. The supernatant from water degradation medium had no detectable protein, showing the stability of the albumin hydrogel in an aqueous medium within the period of our study. (Not shown.)

### Example 2 Biocompatibility and Toxicity of HSAMA Macromer and Hydrogels

Albumin is a biocompatible and bioactive protein. Modification of albumin with methacrylate groups may change its biocompatibility profile, considering the fact that most methacrylate groups are generally considered to induce cytotoxicity. Unreacted macromer may leach out and induce toxicity during cell-culture studies. Moreover, during cell encapsulation and 3D printing, cells are in brief contact with the macromers, which may reduce cell viability. The cytocompatibility of the HSAMA macromers using the HMC3 human microglial cell line, immortalized using the SV40 virus, was investigated. This cell line responds well to different pro-inflammatory stimuli and was used to investigate the toxicity as well as the inflammatory response of both the HSAMA macromer and the resulting hydrogel.

Biocompatibility of HSAMA macromers: The cytocompatibility of both HSAMA macromers as well as HSAMA hydrogel was evaluated using the human microglial HMC3 cell line (ATCC). Cells were cultured in complete EMEM media containing Eagle's minimum essential medium (EMEM) (ATCC® 30-2003™) and 10% fetal bovine serum (FBS, Gibco, Life Technologies) and 1% antibiotic Penicillin-Streptomycin (Gibco, Life Technologies) in a humified atmosphere at 37° C. in 5% CO₂. The medium was changed every 2 days. For cytotoxicity evaluation of HSAMA macromers, first, they were dissolved in complete medium at a concentration of 10, 5, 2.5, 1.25, and 0.625 mg/mL and incubated for 30 min to reach 37° C. The HSAMA cells were seeded on 96-well plates at a density of 7500 cells/well one day prior to the experiment and allowed to attach to the plates for 24 h. On the day of the experiments, the medium was removed, and the 100 μL complement medium containing serial dilution of HSMA macromer was added to the cells with six replicates. After 24 h, cell viability was assessed using CyQUANT™ XTT Cell Viability Assay (Invitrogen™) according to the manufacturer's instructions. Briefly, the cell culture medium was removed and replaced with a serum-free medium. Then XTT reagent was thawed at 37° C., and following mixing with an electron coupling reagent at room temperature, 70 μL of the prepared working solution was directly added to each test well of the 96-well plate. Following incubation at 37° C. for 4 h, absorbance at 450 nm and 660 nm was read on a plate reader (SpectraMax© ID5, Molecular Devices), and specific absorbance was calculated. Pure HSA samples and deionized sterile water were used as negative and positive controls, respectively.

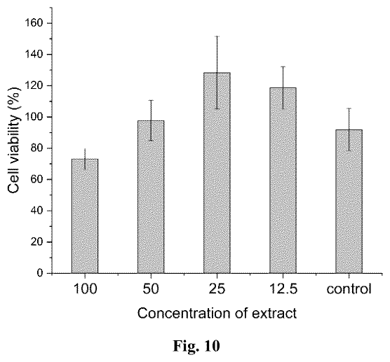

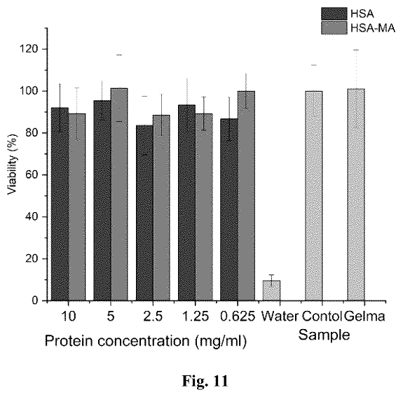

Toxicity and mitochondrial activity were assessed after adding the HSAMA macromer and HSA at various concentrations ranging from 10 mg/ml to 0.625 mg/mL (FIGS. 10 and 11). Overall, there was no significant difference between the cell viability of HSA and corresponding HSAMA at the studied concentrations. For comparison, we also investigated the toxicity of gelatin methacryloyl (gelMA) macromer, a well-established macromer for cell encapsulation and hydrogel preparation. Interestingly, there was no significant difference in cell viability among control, gelMA, and HSA and its methacrylated derivative.

To evaluate the morphology of HMC3 cells treated with both HSA and HSAMA, cells were seeded at a density of 2×10⁴ cell/well in glass chambers (Lab-Tek II Chamber Slide System, Thermo Scientific) and incubated for 24 h at CO₂ incubator at 37° C. The HSA and HSAMA extracts at the concentrations mentioned above were added to the cells. After 24 h incubation at 37° C., the cells were fixed with 4% paraformaldehyde for 15 min at room temperature, and then the cytoskeleton and nuclei were stained. The fixed cells were washed with PBS and permeabilized with 0.1% Triton X-100 for 5 min. The cells were then stained with 10 mM diamidino-2-phenylindole (DAPI) solution in water (Abcam, USA) and Phalloidin-iFluor 488 (Abcam, USA). The morphology of cells was evaluated using an Olympus FluoView FV1000 confocal laser scanning microscope (Olympus Corporation, Toyko, Japan) configured on an inverted Olympus IX81 microscope using either an Olympus 10× UPlanFL N dry objective (NA 0.30), a 20× UPlanFL N dry objective (NA 0.50) or a 40× UPlanFL N oil objective (NA 1.30). DAPI-labeled nuclei in the fixed cells were imaged using a 405 nm diode laser for excitation, and blue fluorescence was recorded using a 430-470 nm band pass emission filter. Bodipy-Phalloidin-labeled actin filaments were imaged using a 488 nm laser for excitation, and green fluorescence was recorded using a 505 nm long-pass emission filter.

HSAMA was well tolerated by the HMC3 cells, and there was no significant difference in terms of morphology among control and HSA- and HSAMA-treated cells. (Data not shown.) HMC3 cells were mostly globular and elongated, and compared to either HSA or HSAMA, there was no difference in the actin cytoskeleton structure. Generally, healthy HMC3 cells have been reported to show a combination of globular, bipolar, and elongated morphology. These morphological studies also confirmed the nontoxic nature of modified HSA at the studied concentrations.

Biocompatibility of HSAMA hydrogels: Similarly, the viability, proliferation, and morphology of HMC3 cells grown on top of hydrogels were evaluated. Prepared HSAMA hydrogel disks (D: 5 mm, H: 5 mm) were placed in complete culture medium at concentration of 0.2 g/mL and incubated at 37° C. for 24 h. HMC3 cells were seeded at a density of 7.5×10³ cells/well in 96-well plates and cultured for 24 h. On the day of the experiment, the cell culture medium was replaced with the extract solution of hydrogels with no dilution (100%) and 50, 25, 12.5, and 6.25% dilution with complete medium with six replicates. After 24 h culture, viability was assessed using CyQUANT™ XTT Cell Viability Assay (Invitrogen™) as described above.

For evaluation of cell attachment and proliferation on top of HSAMA hydrogels, 100 μL of HSAMA macromer (15%, w/w) and LAP initiator (1% of macromer, w/w) in PBS was cast on glass chambers (Lab-Tek II Chamber Slide System, Thermo Scientific) and then photocrosslinked for 10 min as described. It was ensured that the HSAMA hydrogels covered all the glass surfaces in each chamber. The gels were then soaked with a complete medium at 37° C. for 24 h. The medium was then removed, and cells at a density of 2.5×10⁴ cell were seeded on the top of the hydrogels. Cells were then cultured for 1, 3, and 5 days with media refreshment every two days. Cytoskeleton and nuclei were stained and imaged using confocal microscopy. Confocal images were collected using an Olympus FluoView FV1000 confocal laser scanning microscope (Olympus Corporation, Tokyo, Japan) configured on an inverted Olympus IX81 microscope using either an Olympus 10× UPlanFL N dry objective (NA 0.30) or a 20× UPlanFL N dry objective (NA 0.50). Nuc650 (Biotium, CA, USA)-labeled nuclei in the fixed cells were imaged using a 633 nm helium-neon gas laser for excitation, and far-red fluorescence was recorded using a 650 nm long-pass emission filter. Bodipy-Phalloidin-labeled actin filaments were imaged using a 488 nm laser for excitation, and green fluorescence was recorded using a 505 nm long-pass emission filter. Confocal images were recorded through the depth of the sample in 10 μm increments for the 10× objective or 5 μm increments for the 20× objective and displayed as Maximum Intensity Projection images. Empty glass slide chambers with no gel were used as controls.

Cellular attachment to the surface of the biomaterials is crucial for cell migration, viability, and proliferation. Cells that appropriately adhere to the substrate will provide a healthy environment that promotes tissue integration. Attachments and the spreading of immune cells such as the macrophage to the biomaterial's surface is a critical factor in the inflammatory and wound healing responses, ultimately determining the fate of the biomaterial. For example, macrophages can produce a range of chemicals ranging from ROS damaging or eroding the surface to secreting chemotactic cytokines such as interleukin 1, contributing to chronic inflammation, fibrous capsulation, and foreign body reaction.

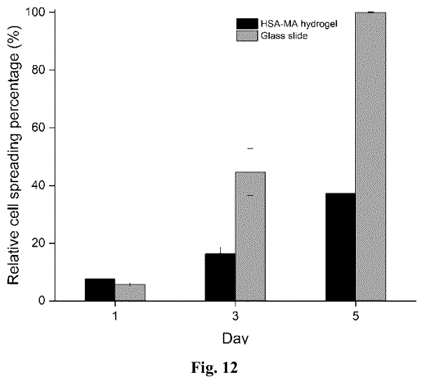

The attachment of HMC3 cells to the surface of the HSAMA gels and control glass slide at days 1, 3, and 5 of culture was evaluated by confocal microscopy evaluated. HMC3 cells on the hydrogels were mostly globular with a few elongated cells with a migratory phenotype. The cells on the tissue culture-treated glass slide were more elongated with a visible F-actin filament network at day 1 of culture. Generally, healthy HMC3 cells show a combination of globular, bipolar, and elongated morphology. The glass slide groups showed typical HMC3 morphologies, while the cells in hydrogel groups covered only a few spots on the surface of the gels. These cells were mainly clumped together and did not spread, unlike the glass slide group 3- and 5-days post-seeding. (FIG. 12.)

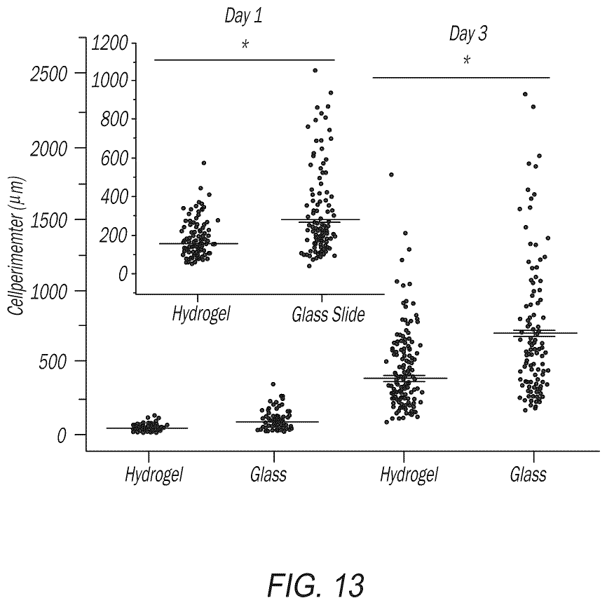

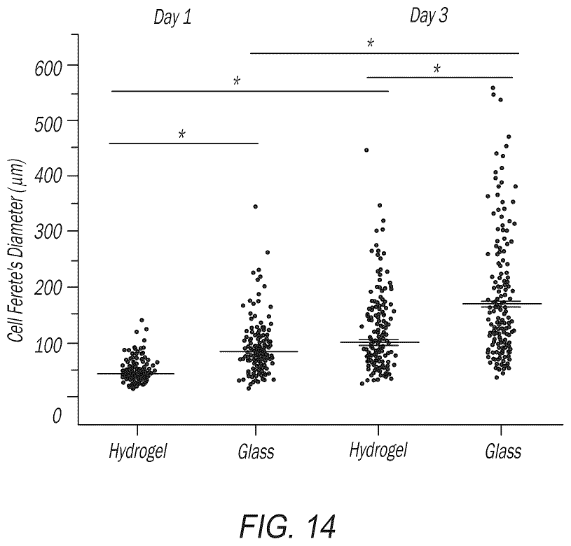

To quantitate cell adhesion to albumin hydrogels, cell perimeters and cell Feret diameters were measured. (FIGS. 13 and 14, respectively.) On day 1 after seeding, HMC3 cells were mainly round with narrow Feret diameter distribution and an average Feret diameter of 50 μm. HMC3 cells on hydrogels had a much smaller average perimeter (150 μm vs. 300 μm). Cells grown on the glass slide had larger Feret diameter distribution, indicating that the surface properties of the glass slides are favorable for HMC3 attachment. After 3 days' culture, quantification of the number of adherent microglia on the surface indicated the HMC3 cells on the surface of the hydrogel grew and the average cells' Feret diameter and perimeters increased to 105 and 400 μm respectively. However, compared to cells grown on glass, cells grown on hydrogel remained smaller, and ultimately, after 5 days post-seeding, achieved <40% confluence.

Biomaterial-cell attachment is mediated through contact with or anchoring to focal contacts, which are mainly heterodimeric transmembrane proteins that contain α and β subunits called integrins. Integrin mediates the cell attachments to the surface of biomaterials by linking the cell membrane to the discrete peptide regions of the ECM component such as collagen or fibronectin. Unlike ECM molecules, which are designed for cell attachment and have an inherent mechanism for recognition and binding to specific anchoring proteins, HSA is not considered a cell-adhesive molecule. The major receptors involved in HSA attachments or internalization are related to albumin hemostasis.

According to the cell adhesion results, it appears that albumin hydrogels can support cell adhesion and proliferation to some extent, despite the absence of structural adhesive molecules such as fibronectin or peptide fragments such as RGD in the network. However, cell attachment was significantly lower in the HSAMA hydrogel compared to the control glass slide. Cell proliferation and spreading doubled every two days, and some HMC3 cells preserved their elongated morphology upon spreading on the top of albumin hydrogels. HMC3 macrophages developed filopodia after five days in culture, which indicate the macrophage's attachments to the surface and its probing the surrounding environment (Data not shown).

The results shown above demonstrate that HMC3 macrophages seeded onto HSAMA hydrogel or plain glass slide controls developed filopodia, which are indicative of the macrophages' surface attachments and ability to interact with the surrounding environment. These findings are corroborated by other studies supporting the idea that low protein binding surfaces such as BSA coatings decrease the attachment of the brain resident macrophages called microglial cells. See Leung et al., Biomaterials, 2020, 29(23), 3289-3297. Although albumin coatings can be effective, the physical nature of surface absorption and the instability of the coating in the presence of biological fluids can limit its practical applications. Crosslinked albumin, on the other hand, is stable and possesses tunable physical and chemical properties that can effectively prevent or reduce macrophage attachment.

### Example 3: Measurement of Cytokine Expression

The inflammatory and wound healing responses to biomaterials is determined by the extent of macrophage adhesion and their cytokine-secretory capabilities. To further characterize macrophage behavior on the surface of the hydrogel, the composition of the cytokines and chemokines produced by the HMC3 macrophages was analyzed with multiplex analysis.

Multiplex analysis of cytokines: Multiplex cytokine assay was used to evaluate the foreign body reaction and inflammatory response to HSAMA macromers and hydrogels. The cell culture supernatant for HSAMA macromers and hydrogels after 24 h culture were analyzed for the presence and concentration of an array of secreted cytokines and growth factors. For each experiment, the cell culture supernatant of six wells was pooled in 2-mL vials and, after centrifugation at 4000 rpm for 10 min, 75 μL of medium from the middle of the centrifugation vial was analyzed. Luminex xMAP technology was used for multiplexed quantification of 48 human cytokines, chemokines, and growth factors. The multiplexing analysis was performed using the Luminex™ 200 system (Luminex, Austin, TX, USA) by Eve Technologies Corp. (Calgary, Alberta). Forty-eight markers were simultaneously measured in the samples using Eve Technologies' Human Cytokine 48-Plex Discovery Assay® (MilliporeSigma, Burlington, Massachusetts, USA) according to the manufacturer's protocol. The 48-plex consists of sCD40L, EGF, eotaxin, FGF-2, FLT-3 ligand, fractalkine, G-CSF, GM-CSF, GROα, IFNα2, IFNγ, IL-1α, IL-1β, IL-1RA, IL-2, IL-3, IL-4, IL-5, IL-6, IL-7, IL-8, IL-9, IL-10, IL-12(p40), IL-12(p70), IL-13, IL-15, IL-17A, IL-17E/IL-25,IL-17F, IL-18, IL-22, IL-27, IP-10, MCP-1, MCP-3, M-CSF, MDC, MIG/CXCL9, MIP-1α, MIP-1β, PDGF-AA, PDGF-AB/BB, RANTES, TGFα, TNFα, TNFβ, and VEGF-A. Assay sensitivities of these markers range from 0.14-50.78 pg/mL for the 48-plex. An average of two readings was reported for each cytokine or chemokine value for the hydrogels group.

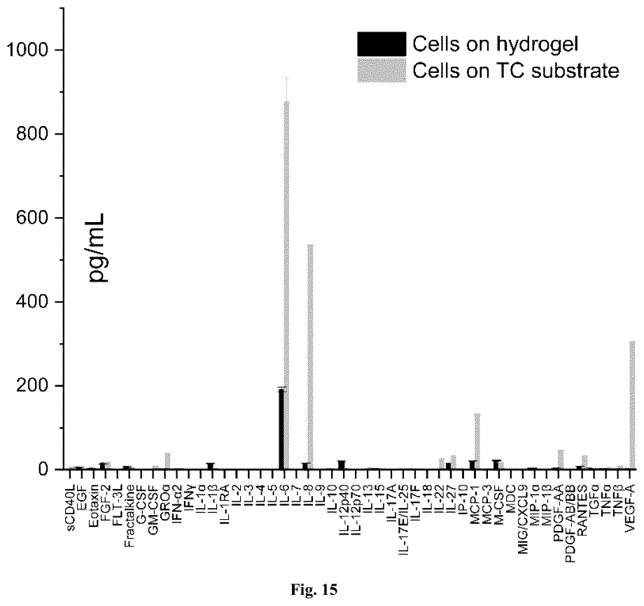

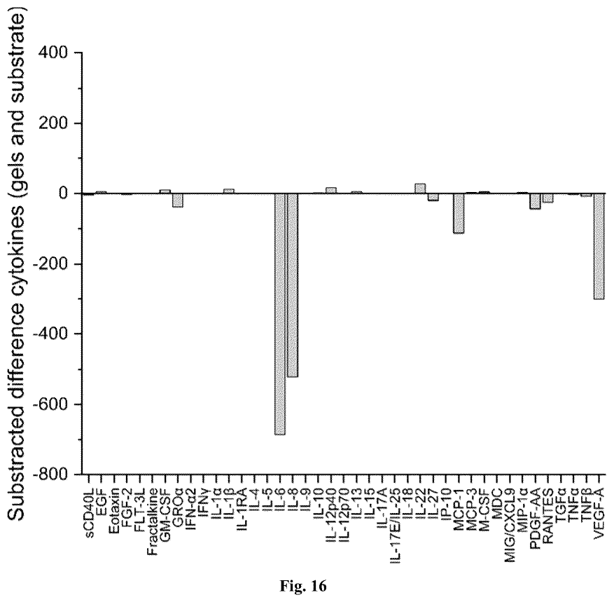

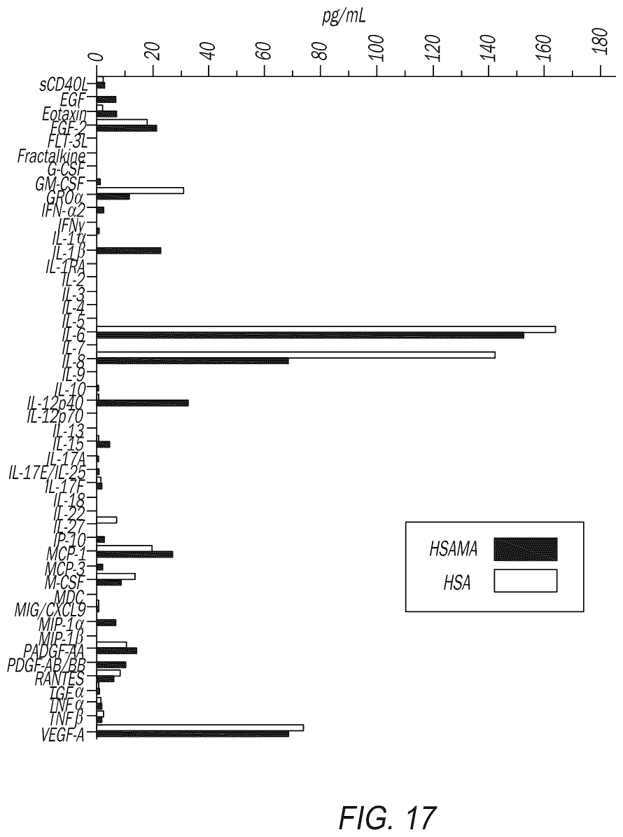

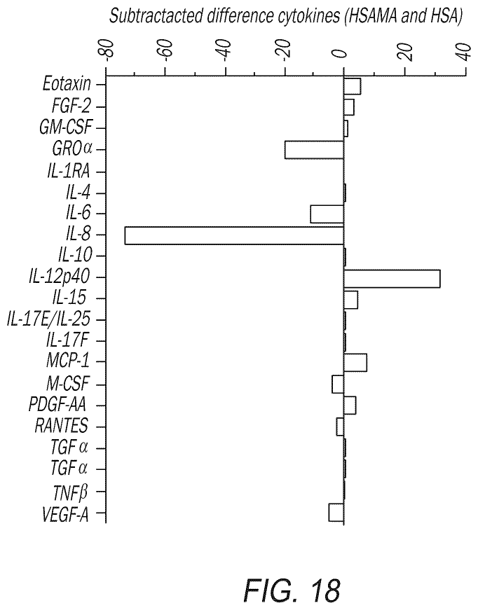

The differential expression of cytokine between groups can also be seen in FIGS. 15-18. The level of 10 major cytokines released by HMC3 into the media on the hydrogel was (in order of decreasing concentration) IL-6>M-CSF>MCP-1>IL-12p40>FGF-2>IL-8>IL-27>IL-1β>RANTES>EGF. The level of 10 major cytokines released into the media by cells cultured on glass was IL-6>IL-8>VEGF-A>MCP-1>PDGF-AA>GROα>IL-27>RANTES>IL-22>FGF-2.

Macrophages exposed to inflammatory stimuli such as exogenous compounds on biomaterial surfaces secrete pro-inflammatory cytokines such as TNF, IL-1, IL-6, IL-8, and IL-12, II-18, IL-23 and Il-27. In this study, the glass slide group elicited a significantly higher (p<0.05) release of IL-6, IL-8, and TNF a. The amount of other key cytokines involved in the inflammatory reaction was also high in the plain glass slide group. For example, high levels of the chemoattractant cytokine MCP-1 were detected in glass slides groups (132 pg/mL). MCP-1 is one of the key chemokines that regulate migration and infiltration of monocytes/macrophages. Interestingly, MCP-1 was reduced to 20 μg/mL for hydrogel groups, suggesting that cells on hydrogels are less capable of recruiting immune cells such as other macrophages or leukocytes towards the hydrogel, which could result in less severe inflammatory response. The levels of VEGF and PDGF in the glass slide group were also much higher than in the albumin hydrogel-coated group (303 pg/mL vs 2.5 pg/mL). These two cytokines are associated with the vascularization process and blood vessel formation. The high concentrations of these cytokines along with high amounts of IL8 and IL 6 indicate high vascular activity, possibly facilitating angiogenesis at the site of inflammation. The macrophages on the surface of the hydrogel were generally smaller in size compared to the glass groups, and had a round rather than spread shape as confirmed by morphology analysis (Data not shown). Macrophages with spherical morphology and a less spread shape have a less developed cytoskeletal organization especially at day 1 and are believed to be less active in an inflammatory environment compared to more spread cells with visibly organized cytoskeletons. This lower macrophage activity may ultimately improve the interaction of the materials with cells, resulting in less perception by inflammatory cells and mild or minimum foreign body reaction.

Interestingly, the addition of unpolymerized albumin methacrylate macromers also reduced the activity of two potent neutrophil chemoattractants, IL8 and growth-related oncogene (GRO) alpha. These chemokines, especially GRO alpha, are involved in wound healing, inflammation, angiogenesis, and tumorigenesis. Long-term elevated expression of these chemokines can result in tissue damage and even elevated angiogenesis and tumorigenesis. It also appears that the HSAMA macromer can influence the production of another chemokine, eotaxin, which is a potent chemoattractant for eosinophils. Eosinophils are an important type of immunomodulatory cell capable of producing and releasing a wide range of cytokines and chemokines such as IL-10 and TGF-β, which can suppress the inflammatory cascade.

Examination of HSAMA hydrogels microstructure using SEM: The microstructures of HSAMA hydrogels were examined by scanning electron microscopy, using a JEOL 7500F (field emission emitter) (JEOL Ltd., Tokyo, Japan). Water in hydrogel samples was first replaced by ethanol through immersing pieces in a series of aqueous ethanol solutions in the range 25-90% followed by immersion in pure ethanol. The samples were then dried using a critical point dryer (Leica Microsystems, Vienna, Austria) using carbon dioxide as the transitional fluid. Program 13 was used, which allowed medium C02 in charge and slow C02 discharge to preserve the micropore structures. Samples were coated with Iridium with a thickness of circa 5.4 nm using Quorum Technologies/Electron Microscopy Sciences Q150T turbo pumped sputter coater (Quorum Technologies, Laughton, East Sussex, England BN8 6BN) purged with argon gas. Samples were imaged at 5 kV.

### Example 4: Measurement of Cytokine Expression

Angiogenesis, the development of new blood vessels from a pre-existing vascular plexus, is a regulated, complex biological process that depends on integrating cellular proliferation, differentiation, and migration. Angiogenesis is required for normal growth and wound healing, and any dysregulated or pathological angiogenesis could result in several diseases such as impaired wound healing and cancer.

The biocompatibility and modulatory effect on angiogenesis of HSAMA and HSAMA-containing hydrogels were investigated in vivo using a chicken embryo model. The highly vascularized extraembryonic membrane (CAM) and vascularized yolk sac were directly exposed to a solution of HSAMA or a disc of HSAMA polymerized hydrogel. The mortality of the treated embryos and the development of blood vessels were evaluated daily for four days. Indicators of angiogenesis such as increased blood vessel density, total blood vessel length, and mean lacunarity were evaluated and quantified for the areas exposed to HSAMA or HSAMA gel.

In vivo Angiogenesis study using the chorioallantoic membrane (CAM) of chicken embryos: The biocompatibility and angiogenesis effect of HSAMA solution and HSAMA gel were studied using the CAM of chicken embryos. Fertilized chicken eggs were bought from the poultry production at Michigan State University and incubated at 37° C. with 55% humidity in an egg incubator (GQF 1500 Digital Cabinet Egg Incubator, Incubator Warehouse, USA). All procedures were ethically approved by the Institutional Animal Care and Use Committee at Michigan State University.

Embryos were divided into three groups and treated on day five of incubation (E5) as follows: the first group (n=22) received 25 μL of PBS and served as control, the second group (n=24) received HSA-MA gel, and the third group (n=8) received 25 μL of HSA-MA solution (15 mg/mL).

The surface of the eggs was cleaned with alcohol; then, a circular incision was made on the top of the eggs manually. A few drops of PBS were added, and the thin outer membrane was carefully removed to prevent vessel bleeding. PBS and HSAMA solution were applied on small glass coverslips and exposed directly to the CAM. HSAMA gel was applied as a small round disk (diameter ˜9 mm, thickness ˜1 mm) directly onto the CAM. The treated eggs were sealed gently and incubated at 37° C. with 55% humidity for four days after treatment. The vascular development of the CAM was examined after 48 h of exposure using a stereomicroscope (AmScope, USA) and images were captured using an 18MP Camera (AmScope, USA). The vascular development of the exposed area (EA) of the CAM was compared to the unexposed area for each embryo. Blood vessel area, total length of vessels, and mean lacunarity of the exposed areas were simulated and quantified using AngioTool Software 0.6a and presented as a percentage of the unexposed areas. Analysis parameters related to vessel thickness and analysis were optimized to obtain the best-simulated blood vessel images. The mortality rate was recorded daily.

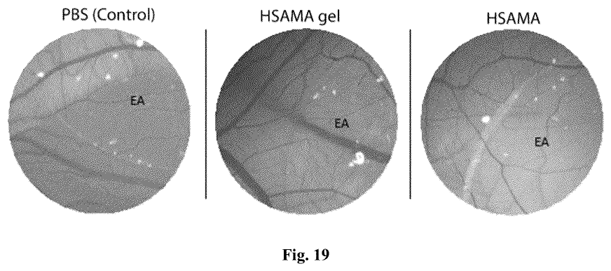

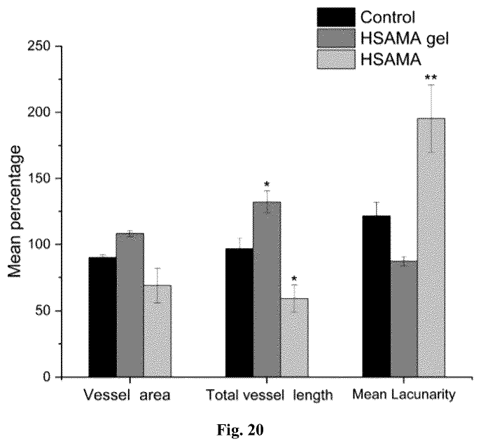

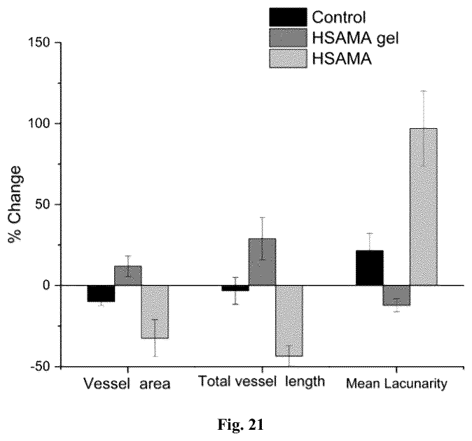

FIG. 19 displays microscopic images of blood vessels after treatment with HSAMA solution or HSAMA hydrogel compared to control. Blood vessels exposed to HSAMA gel showed no evidence of toxicity (such as blood vessel bleeding) and demonstrated a slightly enhanced development of fine blood vessels in the exposed area. Furthermore, chicken embryos treated with HSAMA gel had survival rates similar to untreated embryos after four days of treatment (82.6% vs. 87.5%, respectively). Quantification of the effect of angiogenesis modulation was performed using AngioTool software and revealed a significant increase in the mean percentage of the total blood vessel length when compared to the control (133.3% vs. 96.7%, respectively, p<0.05) (FIGS. 20 and 21).

Corroborated by in vitro toxicity test results, the HSAMA solution demonstrated no evidence of toxicity; however, signs of inhibited angiogenesis were observed (FIG. 9). The survival rate of chicken embryos treated with HSAMA solution was similar to that of embryos treated with HSAMA hydrogel or PBS after four days of treatment (Data not shown). Quantification of angiogenic parameters revealed a significant decrease in total blood vessel length of the exposed area compared to control (60.1% vs. 96.7%, respectively, p<0.05). There was also a significant increase in mean lacunarity compared to control (197.0% vs. 121.5%, respectively, p<0.05) (FIGS. 20 and 21).

The use of the terms “a” and “an” and “the” and “at least one” and similar referents in the context of describing the fusion peptide and related uses (especially in the context of the following claims) are to be construed to cover both the singular and the plural, unless otherwise indicated herein or clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Particular embodiments of the fusion peptide are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the fusion peptide to be practiced otherwise than as specifically described herein. Accordingly, the fusion peptide described herein includes all modifications and equivalents of the subject matter recited in the claims appended hereto as permitted by applicable law. Moreover, any combination of the above-described elements in all possible variations thereof is encompassed by the described fusion peptide unless otherwise indicated herein or otherwise clearly contradicted by context.

1. A hydrogel composition comprising a methacrylated protein, wherein the degree of methacryloylation of the methacrylated protein is greater than about 85%.

2. The hydrogel composition according to claim 1, wherein the protein is selected from the group consisting of albumin, collagen, keratin, silk, gelatin, fibrin, and tropoelastin.

3. The hydrogel composition according to claim 2, wherein the protein is albumin.

4. The hydrogel composition according to claim 3, wherein the albumin is human serum albumin.

5. The hydrogel composition according to claim 1, wherein the hydrogel comprises between about 5% and about 25% (w/w) of the methacrylated protein.

6. The hydrogel composition according to claim 5, wherein the hydrogel comprises between about 10% and about 20% (w/w) of the methacrylated protein.

7. The hydrogel composition according to claim 6, wherein the hydrogel comprises about 11% (w/w), about 15% (w/w), or about 18% (w/w) of the methacrylated protein.

8. (canceled)

9. The hydrogel composition according to claim 1, wherein the degree of methacryloylation of the methacrylated protein is greater than about 90%.

10. The hydrogel composition according to claim 9, wherein the degree of methacryloylation of the methacrylated protein is greater than about 95%.

11. The hydrogel composition according to claim 10, wherein the degree of methacryloylation of the methacrylated protein is greater than about 99%.

12. The hydrogel composition according to claim 11, wherein the degree of methacryloylation of the methacrylated protein is 100%.

13. The hydrogel composition according to claim 2 configured for tissue augmentation.

14. (canceled)

15. The hydrogel composition according to claim 13, wherein the tissue augmentation is selected from the group consisting of treatment of lipodystrophy, repair of vocal cords, and enhancement of tissue sphincters.

16. (canceled)

17. The hydrogel composition according to claim 14, wherein the cosmetic effect is selected from the group consisting of enlarging lips, replacing age-related subcutaneous fat loss, removing wrinkles, as a dermal filler.

18. The hydrogel composition according to claim 1, configured for passivation coatings of a biomedical device, depot, or insert.

19. The hydrogel composition of according to claim 1, further comprising one or more additional pharmaceutical agents.

20. The hydrogel composition according to claim 19, wherein the additional pharmaceutical agent is selected from the group consisting of an anti-inflammatory cytokine, an antibody, an antigen-binding fragment, an anti-inflammatory agent, an NSAID, an anti-angiogenic agent, and combinations thereof.

21. The hydrogel composition according to claim 1, wherein the hydrogels are in the form of microparticles.

22. The hydrogel composition according to claim 21, wherein the microparticles are irregularly shaped.

23. The hydrogel composition according to claim 21, wherein the microparticles are between about 1 and about 100 μm in diameter.

24. The hydrogel according to claim 21, wherein the microparticles have multimodal distribution.

25. A method for modulating an immune response in a subject comprising administering the hydrogel composition a subject in need thereof the hydrogel composition comprising a methacrylated protein, wherein the degree of methacryloylation of the methacrylated protein is greater than about 85%.

26. The method according to claim 25, wherein the hydrogel composition reduces expression of IL-6, IL-8 and/or VEGF.

27. A hydrogel composition comprising a methacrylated protein prepared by
dissolving an unmethacrylated protein in a buffered solution;
adding methacrylic anhydride to the buffered solution to prepare a reaction mixture;
incubating the reaction mixture to generate the methacrylated protein;
isolating the methacrylated protein;
freeze-drying the methacrylated protein;
dissolving the freeze-dried methacrylated protein in deionized water to prepare a hydrogel precursor solution;
adding a photoinitiator to the hydrogel precursor solution and mixing; and
exposing the hydrogel precursor solution to ultraviolet light to cross-link the methacrylated protein to prepare the hydrogel composition,
wherein the ultraviolet intensity is between about 1 and about 20 mW/cm².

28. The hydrogel composition according to claim 27, wherein at least 20% of functional groups in the unmethacrylated protein reactive towards the methacrylic anhydride are reacted.

29. The hydrogel composition according to claim 28, wherein at least 60% of functional groups in the unmethacrylated protein reactive towards the methacrylic anhydride are reacted.

30. The hydrogel composition according to claim 29, wherein at least 80% of functional groups in the unmethacrylated protein reactive towards the methacrylic anhydride are reacted.

31. The hydrogel composition according to claim 30, wherein all functional groups in the unmethacrylated protein reactive towards the methacrylic anhydride are reacted.

32. The hydrogel composition according to claim 27, wherein the protein is selected from the group consisting of albumin, collagen, keratin, silk, gelatin, fibrin, and tropoelastin.

33. The hydrogel composition according to claim 32, wherein the protein is albumin.

34. The hydrogel composition according to claim 27, wherein the incubation took place at room temperature and at a neutral or slightly basic pH.

35. The hydrogel composition according to claim 27, wherein the methacrylated protein was isolated by dialysis in deionized water.

36. The hydrogel composition according to claim 27, wherein the photoinitiator is selected from the group consisting of lithium phenyl-2,4,6-trimethylbenzoylphosphinate, 2-hydroxy-4′-(2-hydroxyethoxy)-2-methylpropiophenone, or ascorbic acid.

37. (canceled)

38. The hydrogel composition according claim 27, wherein the ultraviolet intensity is between about 6 and about 10 mW/cm².

39. The hydrogel composition according claim 38, wherein the ultraviolet intensity is about 6 mW/cm².

40. The hydrogel composition according to claim 27, wherein the deionized water is provided at an amount sufficient to prepare a hydrogel comprising between about 5% and about 25% (w/w) of the methacrylated protein.

41. The hydrogel composition according to claim 38, wherein the hydrogel comprises about 11% (w/w), about 15% (w/w), or about 18% (w/w) of the methacrylated protein.
